(19) United States
(12) Patent Application Publication
(10) Pub. No.: US 2023/0372370 A1
(43) Pub. Date: Nov. 23, 2023

(54) USE OF CANNABINOIDS IN THE TREATMENT OF COVID-19

(71) Applicant: Orcosa Inc., Ewing, NJ (US)

(72) Inventors: Mark RIDALL, Skillman, NJ (US); Vincent T MILETO, Flemington, NJ (US)

(73) Assignee: Orcosa Inc., Ewing, NJ (US)

(21) Appl. No.: 18/317,964

(22) Filed: May 16, 2023

Related U.S. Application Data
(60) Provisional application No. 63/342,934, filed on May 17, 2022.

(51) Int. Cl. A61K 31/00; A61K 9/00; A61K 47/42; A61K 47/26; A61K 45/06

(52) U.S. Cl. CPC A61K 31/658; A61K 9/006; A61K 47/42; A61K 47/26; A61K 45/06

(57) ABSTRACT

A method of treating COVID-19 in a subject, whereby the subject in need thereof is administered, via the oral mucosa, a bioefficient rapidly infusing composition that includes (a) a pharmaceutically acceptable binder and/or excipient system containing gelatin and a sugar alcohol, and (b) a therapeutically effective amount of cannabidiol (CBD) or a derivative/analog thereof.

### CROSS-REFERENCE TO RELATED APPLICATION

The present application claims the benefit of priority to U.S. Provisional Application No. 63/342,934, filed May 17, 2022, which is incorporated herein by reference in its entirety.

### BACKGROUND OF THE INVENTION

### Technical Field

The present disclosure relates to methods of treating respiratory coronavirus SARS-CoV-2 infection (“COVID-19”) with a rapidly infusing composition formulated with cannabidiol (CBD), cannabinoids, or a derivative/analog thereof as the active therapeutic ingredient (ATI).

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Infection by a novel coronavirus, Severe Acute Respiratory Syndrome Coronavirus 2 (SARS-CoV-2), causes the contagious and deadly disease COVID-19, which has circulated as a global pandemic. Symptoms of COVID-19 are generally mild and include fever, cough, fatigue, shortness of breath, muscle aches, and loss of taste and/or smell. These symptoms usually resolve with minimal medical care in a few weeks. However, in some cases the disease may be severe, resulting in hospitalization, ventilation, and/or death.

While vaccines have proven effective in preventing COVID-19 or mitigating its symptoms, systemic treatments such as the viral replicase-inhibitor Remdesivir or the antimalarial drug hydroxychloroquine have proven ineffective or only marginally-effective. Monoclonal antibody treatments have been used to varying degrees of success but are expensive and have efficacies that are highly dependent on the particular SARS-CoV-2 strain. Several other drugs had promising in vitro results but subsequently failed in clinical trials. Additionally, antibody treatments and antiviral drugs must be administered at a medical care facility, such as a hospital, which is inconvenient for patients who would otherwise self-isolate at home to minimize the spread of the virus.

It has been recognized that measures taken at home can be powerful tools in decreasing the spread of COVID-19 by enabling early monitoring and treatment of the disease while minimizing public exposure. These measures include the use of at-home tests and accessible oral drugs that can be taken early in the course of the disease. Thus, there is an interest in an accessible, orally-effective, non-toxic, and relatively inexpensive agent to treat COVID-19.

The treatment of early stages of COVID-19 has been recently suggested using an oral liquid dosage of cannabidiol (CBD) in the form of Epidiolex®. See Nguyen, L. C. et al., “Cannabidiol inhibits SARS-CoV-2 replication through induction of the host ER stress and innate immune responses” Science Advances (2022), DOI: 10.1126/sciadv.abi6110-incorporated herein by reference in its entirety. Epidiolex® is currently the only FDA-approved drug containing CBD as the active ingredient. With this particular drug the CBD is formulated in an oral liquid dosage form with inactive ingredients dehydrated alcohol, sesame oil, strawberry flavor, and sucralose for the treatment of epilepsy, specifically for the treatment of seizures associated with Lennox-Gastaut syndrome, Dravet syndrome, or Tuberous Sclerosis Complex.

However, the oral liquid dosage administration of Epidiolex® suffers from a number of disadvantages. For example, treatment with Epidiolex®, typically requires extremely high doses of CBD, with the FDA-approved recommended dosages of CBD being in the range of 5 mg/kg/day to 25 mg/kg/day for the treatment of epilepsy, divided between two daily doses. These extremely high doses are an unavoidable consequence of oral liquid administration, which results in low bioavailability and inconsistent levels of CBD in systemic circulation. Specifically, drugs taken by mouth and swallowed are absorbed first into the blood perfusing the gastrointestinal (GI) tract. The venous drainage from the GI tract is into the blood perfusing the liver, and thus drugs absorbed from the lumen of the GI tract are immediately presented to the liver—the major detoxifying organ of the body—whereby the drugs are metabolized and then returned to the left side of the heart via the hepatic portal vein and sent into systemic circulation. This first pass metabolism through the liver may result in the removal of a substantial portion of an ingested drug and is more pronounced for some drugs than others; in the case of cannabinoids such as CBD, extensive first pass metabolism provides a paltry bioavailability of only about 6 to 11% when ingested orally. This bioavailability is further affected by whether the subject is in a “fasted” or a “fed” state and even the content of the meal for subjects in the “fed” state.

Another disadvantage associated with medications administered thorough oral liquid dosing is inaccurate dosing. To use Epidiolex®, subjects are instructed to fill a 5 mL oral syringe with the required dosage of the oral solution and then to squirt the contents into the mouth. It is recommended to direct the medication against the inside of the cheek because directing the medication towards the back of the mouth may cause choking. Since each mL of Epidiolex® contains 100 mg of CBD, the instructions recognize that many patients will require greater than 5 mL of oral solution during each of the two daily dosing events to reach the recommended dosage amount, requiring multiple loadings and dispensings of the oral syringe. Any inaccuracy in each of the multiple, daily oral syringe loadings and dispensings can result in large variations in the amount of CBD dosed. This process is not only inaccurate, it is cumbersome and generally unpleasant for the patient. Additionally, the oily and foul taste of CBD exacerbates the unpleasant user experience and can result in poor patient compliance when administered orally.

Given the inconsistencies in bioavailability and dosing of CBD using Epidiolex®, it is also difficult to reliably increase or decrease the amount of CBD in systemic circulation in response to patient outcomes. For instance, given the wide range of possible COVID-19 symptoms and their tendency to change over the course of the infection, a patient may respond better to greater or smaller amounts of CBD. However, for the reasons discussed above, a dosage increase of 20% for CBD in an oral liquid dosage form, for example, would not dependably cause a 20% increase of CBD in systemic circulation.

Yet another disadvantage of the oral liquid dosage administration of Epidiolex® is the adverse reactions associated with consuming voluminous amounts of the liquid carrier, specifically sesame oil, needed to meet the high daily dosing requirements of CBD (5 mg/kg/day to 25 mg/kg/day). As a result, taking Epidiolex® is known to cause gastrointestinal ailments such as diarrhea, decreased weight, gastroenteritis, decreased appetite, and abdominal pain/discomfort. Such adverse reactions increase in patients receiving higher doses of Epidiolex® for the treatment of COVID-19.

In view of the foregoing, there exists a need for a bioefficient COVID-19 treatment using CBD based on dosage forms which are bioavailable, are easy to administer, provide for accurate dosing, minimize adverse reactions, and result in high levels of patient compliance.

### SUMMARY OF THE INVENTION

In U.S. provisional application 63/114,194 and U.S. non-provisional application Ser. No. 17/225,738—each incorporated herein by reference in its entirety, the instant inventors described a rapidly infusing composition containing cannabidiol (CBD) as the active therapeutic ingredient (ATI) and corresponding methods for treating pain using that composition. The inventive rapidly infusing composition provides numerous benefits compared to traditional modes of dosing CBD, such as the oral liquid dosing required with Epidiolex®, including, but not limited to: higher bioavailability; more rapid uptake; more accurate dosing; greater convenience; fewer side effects; and superior patient compliance.

As described in more detail below, the Rapid Infusion Technology™ (RITe) platform formulated with CBD or cannabinoids enables rapid infusion of CBD thereof into systemic circulation via the oral mucosa while bypassing the GI tract and hepatic first pass metabolism, and provides for the first time a consistent, repeatable mechanism for the treatment of COVID-19 in a bioavailable unit dosage form for accurate dosing, easy administration, smaller dosages, reduced side effects, and high levels of patient compliance.

Thus, the present invention provides:

(1) A method of treating COVID-19 caused by the SARS-CoV-2 virus infection in a subject, comprising:
administering to the subject in need thereof, via the oral mucosa, a rapidly infusing composition comprising (a) a pharmaceutically acceptable binder and/or excipient system comprising gelatin and a sugar alcohol, and (b) a therapeutically effective amount of cannabidiol (CBD).

(2) The method of (1), wherein the rapidly infusing composition is lyophilized.

(3) The method of (1) or (2), wherein the rapidly infusing composition has a disintegration time of approximately 1 to 30 seconds in deionized water maintained at 37° C.±2° C.

(4) The method of any one of (1) to (3), wherein the rapidly infusing composition has a disintegration time of approximately 1 to 5 seconds in deionized water maintained at 37° C.±2° C.

(5) The method of any one of (1) to (4), wherein the gelatin is present in the rapidly infusing composition in an amount of 10 to 35 wt. %, based on a total weight of the rapidly infusing composition on a dry basis.

(6) The method of any one of (1) to (5), wherein the gelatin is bovine gelatin.

(7) The method of any one of (1) to (6), wherein the sugar alcohol is present in the rapidly infusing composition in an amount of 5 to 35 wt. %, based on a total weight of the rapidly infusing composition on a dry basis.

(8) The method of any one of (1) to (7), wherein the sugar alcohol comprises mannitol.

(9) The method of any one of (1) to (8), wherein the CBD is present in the rapidly infusing composition in an amount of 20 to 70 wt. %, based on a total weight of the rapidly infusing composition on a dry basis.

(10) The method of any one of (1) to (9), wherein the rapidly infusing composition is formulated with a solid form of the CBD.

(11) The method of any one of (1) to (10), wherein the rapidly infusing composition is formulated with a solid form of the CBD having a purity between 95 and 99.9 wt. %.

(12) The method of any one of (1) to (11), wherein the rapidly infusing composition is formulated with a solid form of the CBD that has been micronized to have a D5 diameter between 1 and 50 sm.

(13) The method of any one of (1) to (12), wherein the rapidly infusing composition further comprises at least one selected from the group consisting of a sweetener, a flavorant, and a colorant.

(14) The method of (13), wherein the rapidly infusing composition comprises the flavorant, and the flavorant comprises lemon-lime flavor.

(15) The method of (13) or (14), wherein the rapidly infusing composition comprises the colorant, and the colorant comprises FD&C Yellow #5.

(16) The method of any one of (13) to (15), wherein the rapidly infusing composition comprises the sweetener, and the sweetener comprises a mixture of sucralose and acesulfame-K.

(17) The method of any one of (1) to (16), wherein the rapidly infusing composition is administered to the subject via the buccal mucosa.

(18) The method of any one of (1) to (17), wherein the therapeutically effective amount of CBD is from 0.1 mg/kg/day to less than 7 mg/kg/day.

(19) The method of any one of (1) to (17), wherein the therapeutically effective amount of CBD is from 0.1 mg/kg/day to less than 5 mg/kg/day.

(20) The method of any one of (1) to (19), wherein the rapidly infusing composition is administered to the subject 1 to 3 times per day.

(21) The method of any one of (1) to (20), wherein CBD is the only active therapeutic ingredient in the rapidly infusing composition.

(22) The method of any one of (1) to (21) wherein the subject is not administered a cannabinoid other than CBD.

(23) The method of any one of (1) to (22), wherein the subject presents with at least one symptom selected from the group consisting of ageusia, anosmia, cough, diarrhea, fatigue, fever, headache, migraine headache, joint pain, muscle pain, nasal congestion, parosmia, runny nose, shortness of breath, and sore throat.

(24) The method of any one of (1) to (23), wherein the subject presents with a positive nucleic acid test for SARS-CoV-2.

(25) The method of any one of (1) to (24), wherein the subject presents with a positive antigen test for SARS-CoV-2.

(26). The method of any one of (1) to (25), wherein the subject is asymptomatic.

(27) The method of any one of (1) to (26), wherein the subject is first administered the rapidly infusing composition at a time that is 5 days or less from the start of SARS-CoV-2 infection or 5 days or less from receiving a positive test result indicating SARS-CoV-2 infection.

(28) The method of any one of (1) to 27), wherein the subject is first administered the rapidly infusing composition at a time that is 3 days or less from the onset of a COVID-19 symptom.

(29) The method of any one of (1) to (28), wherein the subject has long COVID.

(30) The method of any one of (1) to (29), wherein the SARS-CoV-2 is a variant selected from the group consisting of Alpha, Beta. Gamma, Delta, and Omicron.

(31) The method of any one of (1) to (30), wherein a severity or a frequency of a COVID-19 symptom is reduced by at least 50%, relative to the frequency or severity observed prior to administration of the rapidly infusing composition.

(32) The method of any one of (1) to (31), wherein a viral load of the subject as measured by nasal secretion is reduced by at least 3-fold, relative to the viral load prior to administration of the rapidly infusing composition.

(33) The method of any one of (1) to (32), wherein a viral load of the subject as measured by lung secretion is reduced by at least 20-fold, relative to the viral load prior to administration of the rapidly infusing composition.

(34) The method of any one of (1) to (33), wherein the rapidly infusing composition is administered in combination with an antiviral drug.

(35) The method of (34), wherein the antiviral drug is at least one selected from the group consisting of amiodarone, artesunate, chlorpromazine, clemastine, elacridar, favipiravir, lopinavir, molnupiravir, nirmatrelvir, pyronaridine, remdesivir, ribavirin, ritonavir, sertraline, triazavirin, and umifenovir.

(36) The method of (34), wherein the antiviral drug is an RNA virus antiviral drug.

(37) The method of (36), wherein the RNA virus antiviral drug is at least one selected from the group consisting of adapromine, amantadine, asunaprevir, baloxavir marboxil, beclabuvir, bemnifosbuvir, boceprevir, bulevirtide, ciluprevir, CMX521, daclatasvir, daclatasvir, danoprevir, dasabuvir, deleobuvir, eicar, elbasvir, elbasvir, faldaprevir, favipiravir, filibuvir, galidesivir, glecaprevir, glecaprevir, grazoprevir, grazoprevir, GS-441524, GS-6620, IDX-184, interferon alfa 2b, laninamivir, ledipasvir, ledipasvir, lufotrelvir, mericitabine, merimepodib, MK-608, molnupiravir, moroxydine, narlaprevir, nirmatrelvir, NITD008, odalasvir, otnbitasvir, ombitasvir, oseltamivir, paritaprevir, paritaprevir, peginterferon alfa-2a, peginterferon alfa-2b, peramivir, pibrentasvir, pibrentasvir, pimodivir, pleconaril, presatovir, radalbuvir, ravidasvir, remdesivir, ribavirin, rimantadine, ritonavir, ruzasvir, samatasvir, setrobuvir, simeprevir, sofosbuvir, sofosbuvir, sofosbuvir, sofosbuvir, sofosbuvir, sovaprevir, taribavirin, tegobuvir, telaprevir, TMC-647055, triazavirin, umifenovir, uprifosbuyir, valopicitabine, vaniprevir, vedroprevir, velpatasvir, velpatasvir, velpatasvir, voxilaprevir, voxilaprevir, and zanamivir.

(38) The method of any one of (1) to (37), wherein the rapidly infusing composition is administered in combination with an angiotensin-converting-enzyme inhibitor.

(39) The method of (38), wherein the angiotensin-converting-enzyme inhibitor is at least one selected from the group consisting of alacepril, benazepril, captopril, ceronapril, cilazapril, delapril, enalapril, fosinopril, imidapril, lisinopril, moexipril, perindopril, quinapril, ramipril, rentiapril, spirapril, temocapril, trandolapril, and zofenopril.

(40) The method of any one of (1) to (39), wherein the rapidly infusing composition is administered in combination with a monoclonal antibody.

(41) The method of (40), wherein the monoclonal antibody is at least one selected from the group consisting of bamlanivimab, bamlanivimab/etesevimab, baricitinib, casirivimab, casirivimab/imdevimab, cilgavimab, etesevimab, imdevimab, lenzilumab, regdanvimab, sarilumab, sotrovimab, tixagevimab, tixagevimab/cilgavimab, tixagevimab/cilgavimab, and tocilizumab

(42) The method of any one of (1) to (41), wherein the rapidly infusing composition is administered in combination with an angiotensin II receptor blocker.

(43) The method of (42), wherein the angiotensin II receptor blocker is at least one selected from the group consisting of azilsartan, candesartan, eprosartan, fimasartan, irbesartan, losartan, olmesartan, tasosartan, telmisartan, and valsartan.

(44) The method of any one of (1) to (43), wherein the subject is asymptomatic and had been exposed to SARS-CoV-2 before the administering.

### DETAILED DESCRIPTION OF THE INVENTION

6 In the following description, it is understood that other embodiments may be utilized and structural and operational changes may be made without departure from the scope of the present embodiments disclosed herein.

### Definitions

As used herein, the terms “compound” and “product” are used interchangeably, and are intended to refer to a chemical entity, whether in the solid, liquid or gaseous phase, and whether in a crude mixture or purified and isolated. Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the disclosure. Many geometric isomers of C═C double bonds, C═N double bonds, ring systems, and the like can also be present, and all such stable isomers are contemplated in the present disclosure. Cis- and trans- (or E- and Z-) geometric isomers, when present, may be isolated as a mixture of isomers or as separated isomeric forms. Compounds referenced in the disclosure can be isolated in optically active or racemic forms. Optically active forms may be prepared by resolution of racemic forms or by synthesis from optically active starting materials. All processes used to prepare these compounds and intermediates made therein are considered to be part of the present disclosure. When enantiomeric or diastereomeric products are prepared, they may be separated by conventional methods, for example, by chromatography, fractional crystallization, or through the use of a chiral agent. Depending on the process conditions, the end products referenced in the present disclosure are obtained either in free (neutral) or salt form. Both the free form and the salts of these end products are within the scope of the disclosure. If so desired, one form of a compound may be converted into another form. A free base or acid may be converted into a salt; a salt may be converted into the free compound or another salt; a mixture of isomeric compounds may be separated into the individual isomers. Compounds referenced in the present disclosure, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the disclosure. Further, a given chemical formula or name shall encompass all conformers, rotamers, or conformational isomers thereof where such isomers exist. Different conformations can have different energies, can usually interconvert, and are very rarely isolatable. There are some molecules that can be isolated in several conformations. For example, atropisomers are isomers resulting from hindered rotation about single bonds where the steric strain barrier to rotation is high enough to allow for the isolation of the conformers. It should be understood that all conformers, rotamers, or conformational isomer forms, insofar as they may exist, are included within the present disclosure.

As used herein, the term “solvate” refers to a physical association of a referenced compound with one or more solvent molecules, whether organic or inorganic. This physical association includes hydrogen bonding. In certain instances, the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. Solvate encompasses both solution phase and isolable solvates. Exemplary solvent molecules which may form the solvate include, but are not limited to, water, methanol, ethanol, n-propanol, isopropanol, n-butanol, isobutanol, tert-butanol, ethyl acetate and other lower alkanols, glycerin, acetone, dichloromethane (DCM), dimethyl sulfoxide (DMSO), dimethyl acetate (DMA), dimethylformamide (DMF), isopropyl ether, acetonitrile, toluene, N-methylpyrrolidone (NMP), tetrahydrofuran (THF), tetrahydropyran, other cyclic mono-, di- and tri-ethers, polyalkylene glycols (e.g., polyethylene glycol, polypropylene glycol, propylene glycol), and mixtures thereof in suitable proportions. Exemplary solvates include, but are not limited to, hydrates, ethanolates, methanolates, isopropanolates and mixtures thereof. Methods of solvation are generally known to those of ordinary skill in the art.

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids and phenols. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two, generally, non-aqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa. (1990)—which is incorporated herein by reference in its entirety.

When referencing a particular composition/material, the phrase “consists essentially of”, means that the particular composition/material may include minor amounts of impurities so long as those impurities do not affect the basic and novel property of the invention—the ability to treat COVID-19.

As used herein, the terms “optional” or “optionally” means that the subsequently described event(s) can or cannot occur or the subsequently described component(s) may or may not be present (e.g., 0 wt. %).

As used herein, the terms “treat”, “treatment”, and “treating” in the context of the administration of a therapy to a subject in need thereof refers to the reduction or amelioration of severity of symptoms of the condition being treated; reduction of duration of symptoms of the condition being treated; reduction, inhibition, slowing, or arresting of the progression of symptoms associated with the condition; reduction of frequency of symptoms of the condition being treated; elimination of symptoms and/or underlying cause of the condition; prevention of the occurrence of symptoms of the condition, for example in a subject that may be predisposed to the condition but does not yet experience or exhibit symptoms of the condition; reducing viral load of SARS-CoV-2; reducing communicability of SARS-CoV-2; inhibiting replication of SARS-CoV-2; improvement or remediation or amelioration of damage following a condition, for example improving, remediating, or ameliorating neurological damage or other physical damage associated with COVID-19 or post-COVID-19 syndrome (“long COVID”); providing a prophylactic or preventative measure against COVID-19 symptoms and/or SARS-CoV-2 infection.

The term “prophylactic” or “preventative” when used refers to the administration of an active compound to prevent, reduce the likelihood of an occurrence or a reoccurrence of COVID-19, or to minimize a new infection of SARS-CoV-2 relative to infection that would occur without such treatment.

The term “subject” and “patient” are used interchangeably. As used herein, they refer to any subject for whom or which therapy is desired. In most embodiments, the subject is a human.

The terms “administer”, “administering”, “administration”, and the like, as used herein, refer to the methods that may be used to enable delivery of the active therapeutic ingredient (ATI) to the desired site of biological action. Routes or modes of administration are as set forth herein.

The term “Rapid Infusion Technology™ (RITe) platform” or “rapidly infusing composition”, as used herein means a solid dosage form containing medicinal substances that disintegrates rapidly in the oral cavity (when contacted with saliva) with no need for chewing or drinking/swallowing liquids (e.g., water, liquid carriers, saliva, etc.) to ingest these medicinal substances, with an in-vitro disintegration time of 30 second or less according to the United States Pharmacopeia (USP) <701> Disintegration Test. The disclosed rapidly infusing compositions are thus a different dosage form than, for example, a chewable tablet, a lozenge intended to be dissolved slowly in the mouth, an orally disintegrating film or tablet designed to be dissolved/disintegrated in the mouth and swallowed (also called “orodispersible” formulations), a tablet that should be swallowed whole with food or liquid, oral liquid dosage forms, or any other oral dosage form designed for absorption from the GI tract.

The dosage amount and treatment duration are dependent on factors, such as bioavailability of a drug, administration mode, toxicity of a drug, gender, age, lifestyle, body weight, the use of other drugs and dietary supplements, the disease stage, tolerance and resistance of the body to the administered drug, etc., and then determined and adjusted accordingly. The terms “effective amount” or “therapeutically effective amount” refer to a sufficient amount of an active therapeutic ingredient (ATI) being administered which provides the desired therapeutic or physiological effect or outcome, for example, the amount of ATI sufficient for reducing the frequency or elimination of COVID-19 symptoms or viral load. The result can be a reduction and/or alleviation of the signs, symptoms, or causes of a disease, or any other desired alteration of a biological system. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the age and general condition of the subject, mode of administration, and the like. An appropriate “effective amount” in any individual case may be determined by one of ordinary skill in the art using only routine experimentation, for example through the use of dose escalation studies.

### Rapid Infusion Technology™ (RITe) Platform

The present disclosure provides a therapeutic formulation presented in the form of a rapidly infusing composition which is suitable for administration of lipophilic active therapeutic ingredients (ATIs) such as cannabidiol (CBD) via a non-gastric mucosal surface. As described in more detail below, the novel delivery platform allows otherwise difficult to formulate ATIs—such as CBD—to be presented in unit dosage form for accurate dosing and in an easy-to-take format for high levels of patient compliance. For example, the rapidly infusing composition may be presented in tablet form and packaged in individual blister units.

In particular, the rapidly infusing composition enables oral mucosal administration of lipophilic ATIs in a solid dosage form directly into systemic circulation via the sublingual mucosa or the buccal mucosa and avoidance of first pass metabolism. The rapidly infusing composition thus presents lipophilic ATIs such as CBD (which may otherwise be susceptible to extensive first pass metabolism) in an enhanced bioefficient dosage form. Such exemplary dosage forms may include, inter alia, enhanced bioavailability, typically with a bioavailability of at least 20%, preferably at least 30%, preferably at least 40%, preferably at least 50%, preferably at least 60%, preferably at least 70%, preferably at least 80%, preferably at least 90%, and up to 95%, preferably up to 90%, preferably up to 80%, preferably up to 70%, preferably up to 60%, preferably up to 50%, preferably up to 40%, preferably up to 30%. Such enhanced bioavailability allows the dosage amount of ATI to be reduced, whilst maintaining the same pharmacological effect. For example, the RITe™ platform allows CBD to be dosed in an amount of 7 mg/kg/day, preferably under 5 mg/kg/day for the treatment of COVID-19.

Additionally, the bioefficient rapidly infusing composition enables a defined dose of ATI to be absorbed via the oral mucosae, prior to the gastric mucosa, thereby presenting a defined and consistent level of ATI into systemic circulation for consistent and reliable pharmacological effects. Consistency in pharmacological effects helps to improve patient adherence during treatment. The aforementioned enhanced levels of bioavailability may be consistently achieved because the RITe™ platform reduces the tendency for enteral oral administration through voluntary or involuntary swallowing by shortening the residence time the ATI spends in the oral cavity. Any amount of ATI (e.g., CBD) that is swallowed would be subject to first-pass metabolism and thus overall lower bioavailability. Swallowing further results in greater variability in the effective amount of dosing, as a result of variability in the amount swallowed and the greater subject variability of bioavailability through first-pass metabolism for the amount swallowed.

Administration may be carried out by simply placing the rapidly infusing composition directly in the buccal cavity (between the cheek and gum) or over the sublingual mucous gland (under the ventral surface of the tongue). Preferred rapidly infusing compositions are those which are lyophilized products formulated for rapid disintegration when placed in such an oral environment for rapid release of the ATI. The rapidly infusing compositions of the present disclosure may have a disintegration time of from approximately 1 second to 30 seconds or less, preferably 25 seconds or less, preferably 20 seconds or less, preferably 15 seconds or less, preferably 10 seconds or less, preferably 5 seconds or less, preferably 3 seconds or less, according to the United States Pharmacopeia (USP) <701> Disintegration Test performed in deionized water maintained at 37° C.±2°. In particular, preferred rapidly infusing compositions are those formulated for oral disintegration in 5 seconds or less, preferably 4 seconds or less, preferably 3 seconds or less, preferably 2 seconds or less, preferably in approximately 1 second, according to the United States Pharmacopeia (USP) <701> Disintegration Test performed in deionized water maintained at 37° C.±2°. A disintegration profile no higher than the above-mentioned upper limit when in intimate contact with a non-gastric mucosal surface provides for rapid absorption of the ATI and short onset times to therapeutic relief.

The easy-to-take oral mucosal dosage form presented herein is a vast improvement over the cumbersome and generally unpleasant oral liquid dosage form of Epidiolex®, which as discussed heretofore, is known to cause gastrointestinal side effects and often requires multiple loadings and dispensings of the oral syringe to meet the prescribed high dosages—a particular problem if administering to children. In contrast, administration of the rapidly infusing composition of the present disclosure is easy, one simply “takes it and it's gone,” with no need for swallowing, thus offering improved patient compliance whether self-administered or administered by a healthcare provider, a care-taker, etc.

Patient compliance may also be improved in terms of temporary abstinence from swallowing, which is often triggered when a patient is presented with foul-tasting oral medications. Any issues related to foul taste may be minimized with the above rapid disintegration times, which reduces the tendency for enteral oral administration through voluntary or involuntary swallowing, and as a result, the aforementioned enhanced levels of bioavailability may be achieved.

The rapid disintegration profile disclosed herein, coupled with the direct introduction of the ATI into systemic circulation through the sublingual mucosa or the buccal mucosa, preferably through the buccal mucosa, provides a more rapid onset of therapeutic effect. For example, has an onset time of under 15 minutes, preferably under 10 minutes, preferably under 5 minutes, preferably under 4 minutes, preferably under 3 minutes, preferably under 2 minutes, preferably under 1 minute. Such short onset times are superior to those which can be obtained with traditional oral dosage forms such as tablets taken with food or liquids, liquid dosage forms, as well as orodispersible dosage forms dissolved by mouth and then swallowed.

The rapidly infusing composition herein generally contains (a) a pharmaceutically acceptable binder and/or excipient system that includes gelatin and a sugar alcohol e.g., mannitol, and optionally one or more of a sweetener, a flavorant, and a colorant; and (b) a therapeutically effective amount of an active therapeutic ingredient such as cannabidiol (CBD).

Pharmaceutically Acceptable Carrier and/or Excipient System

Carriers and/or excipients are ingredients which do not provide a therapeutic effect themselves, but which are designed to interact with, and enhance the properties of the active therapeutic ingredient. In particular, carriers and/or excipients may act as a vehicle for transporting the active therapeutic ingredient from one organ, or portion of the body, to another organ, or portion of the body. The selection of appropriate carrier/excipient ingredients may impact the solubility, distribution, release profile/kinetics, absorption, serum stability, therapeutic onset time, and ultimately the efficacy of the ATI, as well as the shelf-life, dosage forms, and processability of the drug product. Each ingredient in the pharmaceutically acceptable carrier and/or excipient system must be “pharmaceutically acceptable” in the sense of being compatible with the other ingredients of the rapidly infusing composition and not injurious to the patient.

In light of the above, particular preference is given herein to pharmaceutically acceptable carrier and/or excipient systems which include gelatin and a sugar alcohol (e.g., mannitol).

Gelatin is to be included in the pharmaceutically acceptable carrier and/or excipient system in order to effect matrix formation in the lyophilized product, i.e., gelatin may act primarily as a matrix former. During manufacture of the rapidly infusing composition, lyophilization from an aqueous suspension results in the removal of water thereby leaving behind a gelatin matrix/scaffolding/lattice upon which the ATI can be evenly dispersed or suspended. It has been found that gelatin has a propensity to establish a stable matrix in lyophilized form, yet allow for rapid disintegration when brought into contact with the aqueous oral environment, thereby providing efficient transfer of the ATI from the hydrophilic vehicle to the oral mucosa. In this regard, mammalian gelatins such as bovine gelatin and porcine gelatin are preferred, with bovine gelatin being particularly preferred. In some embodiments, the rapidly infusing composition does not contain fish gelatin.

The amount of gelatin used may be varied. Generally, gelatin may be present in the rapidly infusing composition in an amount of at least 10 wt. %, preferably at least 12 wt. %, preferably at least 14 wt. %, preferably at least 16 wt. %, preferably at least 18 wt. %, preferably at least 20 wt. %, preferably at least 22 wt. %, and up to 50 wt. %, preferably up to 45 wt. %, preferably up to 40 wt. %, preferably up to 35 wt. %, preferably up to 32 wt. %, preferably up to 30 wt. %, preferably up to 28 wt. %, preferably up to 26 wt. %, preferably up to 24 wt. %, based on a total weight of the rapidly infusing composition on a dry basis.

The pharmaceutically acceptable carrier and/or excipient system is also formulated with one or more sugar alcohols, which may act primarily as a bulking agent. Examples of sugar alcohols include, but are not limited to, erythritol, xylitol, sorbitol, maltitol, mannitol, lactitol, and glycerin, which may be used singly or in combinations. Advantage can also be taken of the effect of certain sugar alcohols in terms of taste (sweetness and coolness due to endothermal heat of solution), as well as their ability to aid/speed tablet disintegration. In this regard, particular preference is given to mannitol.

The sugar alcohol, preferably mannitol, may be present in the rapidly infusing composition in any amount which provides the desired bulking/taste/disintegration effects. Generally, this amount will range from of at least 5 wt. %, preferably at least 10 wt. %, preferably at least 12 wt. %, preferably at least 14 wt. %, preferably at least 16 wt. %, preferably at least 12 wt. %, and up to 50 wt. %, preferably up to 45 wt. %, preferably up to 40 wt. %, preferably up to 35 wt. %, preferably up to 30 wt. %, preferably up to 28 wt. %, preferably up to 26 wt. %, preferably up to 24 wt. %, preferably up to 22 wt. %, preferably up to 20 wt. %, based on a total weight of the rapidly infusing composition on a dry basis.

In some embodiments, a weight ratio of gelatin to sugar alcohol ranges from 1:3, preferably from 1:2, preferably from 1:1, preferably from 1.1:1, and up to 3:1, preferably up to 2:1, preferably up to 1.5:1, preferably up to 1.2:1.

The pharmaceutically acceptable carrier and/or excipient system may also optionally include one or more of a sweetener, a flavorant, and a colorant.

The sweetener may be used in any amount which provides the desired sweetening effect, generally in amount of 0 to 10 wt. %, for example in an amount of up to 10 wt. %, preferably up to 8 wt. %, preferably up to 6 wt. %, preferably up to 5 wt. %, preferably up to 4 wt. %, preferably up to 3 wt. %, preferably up to 2 wt. %, preferably up to 1.5 wt. %, preferably up to 1 wt. %, preferably up to 0.5 wt. %, preferably up to 0.1 wt. %, based on a total weight of the rapidly infusing composition on a dry basis. Suitable examples of sweeteners include, but are not limited to, aspartame, saccharin (as sodium, potassium or calcium saccharin), cyclamate (as a sodium, potassium or calcium salt), sucralose, acesulfame-K, thaumatin, neohisperidin, dihydrochalcone, ammoniated glycyrrhizin, dextrose, maltodextrin, fructose, levulose, sucrose, and glucose, which may be used singly or in combinations, with particular preference given to sucralose and acesulfame-K.

It is to be readily appreciated by those of ordinary skill in the art that one or more flavorants may be optionally included in the rapidly infusing composition to mask any unpleasant taste imparted by certain ingredients (e.g., an unpleasant tasting ATI) or to otherwise impart an acceptable taste profile to the composition, and the composition is not limited to any particular flavor. Suitable flavorants include, but are not limited to, oil of wintergreen, oil of peppermint, oil of spearmint, oil of sassafras, oil of clove, cinnamon, anethole, menthol, thymol, eugenol, eucalyptol, lemon, lime, lemon-lime, orange, and other such flavor compounds to add fruit notes (e.g., citrus, cherry etc.), spice notes, etc., to the composition. The flavorants may be constitutionally composed of aldehydes, ketones, esters, acids, alcohols (including both aliphatic and aromatic alcohols), as well as mixtures thereof. Specific mention is made to lemon-lime flavor powder, which works particularly well with CBD as the ATI. The flavorant may be used in any amount which provides the desired flavor, generally in an amount of 0 to 10 wt. %, for example in an amount of up to 10 wt. %, preferably up to 8 wt. %, preferably up to 6 wt. %, preferably up to 5 wt. %, preferably up to 4 wt. %, preferably up to 3 wt. %, preferably up to 2 wt. %, preferably up to 1.5 wt. %, preferably up to 1 wt. %, preferably up to 0.5 wt. %, preferably up to 0.1 wt. %, based on a total weight of the rapidly infusing composition on a dry basis.

Two main strategies contribute to the taste masking success of the present disclosure. First, any issues related to foul taste are fundamentally mitigated by the short oral residence times provided by the rapid disintegration profile described heretofore. One “takes it and it's gone.” Second, when formulated with a flavorant, a robust mixture of flavors may hit the tongue at essentially the same time—the flavor of the CBD still may hit the tongue, but the perception of the flavor is canceled or mitigated by the simultaneous arrival of other flavors. Even then, the robust mixture of flavors will quickly subside as the composition is rapidly absorbed through the oral mucosa.

Likewise, the rapidly infusing composition may be colored or tinted through the optional use of one or more colorants. Suitable colorants are those approved by appropriate regulatory bodies such as the FDA and those listed in the European Food and Pharmaceutical Directives and include both pigments and dyes such as FD&C and D&C dyes, with specific mention being made to FD&C Yellow #5.

In addition to gelatin and a sugar alcohol (e.g., mannitol), and optionally one or more of a sweetener, a flavorant, and a colorant, the pharmaceutically acceptable carrier and/or excipient system may optionally include one or more other pharmaceutically acceptable carriers and/or excipients. Examples of which include, but are not limited to,
fillers or extenders such as starches (e.g., corn starch and potato starch), sugars (e.g., lactose or milk sugar, maltose, fructose, glucose, trehalose, sucrose), dextrates, dextrin, polydextrose, high molecular weight polyethylene glycols, silicic acid, potassium sulfate, aluminum monostearate, polyesters, polycarbonates, and polyanhydrides;
binders, such as cellulose and its derivatives, (e.g., carboxymethyl cellulose, sodium carboxymethyl cellulose, hydroxypropyl cellulose, hydroxyethyl cellulose, hydroxypropylmethyl cellulose (hypromellose), hydroxyethyl methyl cellulose, methyl cellulose, ethyl cellulose, cellulose acetate, cellulose acetate phthalate, and microcrystalline cellulose), alginates (e.g., sodium alginate), polyvinyl pyrrolidone, polyvinyl acetate-vinylpyrrolidone, polyacrylic acid, methacrylate copolymers (e.g., methyl methacrylate copolymers and Eudragit® products available from Evonik), modified starch, powdered tragacanth, malt, acacia (gum arabic), carbomer/carboxyvinyl polymer, carrageenan, chitosan, copovidone, cyclodextrins and modified cyclodextrins, guar gum, inulin, pectin (e.g., low viscosity pectin), polycarbophil or a salt thereof, polyvinyl alcohol, pullulan, xanthan gum, casein, protein extracts (e.g., whey protein extract, soy protein extract), rein, levan, elsinan, gluten, locust bean gum, gellan gum, and agar;
disintegrating agents, such as agar-agar, calcium carbonate, tapioca starch, alginic acid, certain silicates, sodium carbonate, sodium starch glycolate, and cross-linked sodium carboxymethyl cellulose (croscarmellose sodium);
surfactants/absorption accelerators/wetting agents/emulsifying agents/solubilizers, including any of the anionic, cationic, nonionic, zwitterionic, amphoteric and betaine variety, such as polyalkylene oxide copolymers (e.g., poloxamers, polyethylene oxide-polypropylene oxide copolymers), sodium lauryl sulfate, sodium dodecyl benzene sulfonate, sodium docusate, sodium lauryl sulfoacetate, alkali metal or ammonium salts of lauroyl sarcosinate, myristoyl sarcosinate, palmitoyl sarcosinate, stearoyl sarcosinate and oleoyl sarcosinate, cetyl alcohol, glycerol monostearate, glycerol oleate, fatty acid mono- and di-esters of glycerol, fatty acid esters of polyethylene glycol, polyoxyethylene sorbitol, fatty acid esters of sorbitan, polysorbates (polyalkolyated fatty acid esters of sorbitan) (e.g., polyoxyethylene sorbitan monostearate, monoisostearate and monolaurate), polyethylene oxide condensates of alkyl phenols, cocoamidopropyl betaine, lauramidopropyl betaine, palmityl betaine, glyceryl monooleate, glyceryl monostearate, fatty alcohols (e.g., cetostearyl and cetyl alcohol), medium chain triglycerides, medium chain fatty acids, polyethoxylated castor oil, polyethoxylated alkyl ethers (e.g., ethoxylated isostearyl alcohols), polyethylene glycols (Macrogols), polypropylene glycols, polyoxyethylene stearates, anionic and nonionic emulsifying waxes, propylene glycol alginates, alcohol-oil transesterification products, polyglycerized fatty acids, propylene glycol fatty acid esters, mixtures of propylene glycol fatty acid esters and glycerol fatty acid esters, sterol and sterol derivatives, sugar esters, lower alcohol fatty acid esters, fatty acids and bile acids and their corresponding salts, ricinoleic acid/sodium ricinoleate, linoleic acid/sodium linoleate, lauric acid/sodium laurate, mono-, di-, and tri-hydroxy bile acids and their salts, sulfated bile salt derivatives, phospholipids, ether carboxylates, succinylated monoglycerides, mono/diacetylated tartaric acid esters of mono- and diglycerides, citric acid esters of mono- and diglycerides, alginate salts, and lactylic esters of fatty acids;
plasticizers such as glycerin fatty acid esters, sucrose fatty acid esters, lecithin (e.g., enzyme modified lecithin), polysorbates, sorbitan fatty acid esters, polyethylene glycol, propylene glycol, triacetin, glycerol oleate, medium chain fatty acids, tributyl citrate, triethyl citrate, acetyl tri-n-butyl citrate, diethyl phthalate, castor oil, dibutyl sebacate, and acetylated monoglycerides;
absorbents, such as kaolin and bentonite clay;
lubricants, such as talc, calcium stearate, magnesium stearate, solid polyethylene glycols, zinc stearate, sodium stearate, stearic acid, ethyl oleate, and ethyl laurate;
controlled release agents such as cross-linked polyvinyl pyrrolidone (crospovidone);
opacifying agents such as titanium dioxide;
buffering agents, including alkaline buffering agents, such as sodium hydroxide, sodium citrate, magnesium hydroxide, aluminum hydroxide, sodium carbonate, sodium bicarbonate, potassium phosphate, potassium carbonate, potassium bicarbonate, calcium phosphate, potassium hydroxide, calcium hydroxide, magnesium oxide, potassium dihydrogen phosphate, sodium dihydrogen phosphate, sodium phosphate, calcium carbonate, magnesium carbonate;
osmotic agents such as sodium chloride, calcium chloride, potassium chloride
diluents/tableting agents such as dicalcium phosphate and colloidal silicon dioxide;
antioxidants, including (1) water soluble antioxidants, such as ascorbic acid, cysteine hydrochloride, sodium bisulfate, sodium metabisulfite, and sodium sulphite, (2) oil-soluble antioxidants, such as ascorbyl palmitate, butylated hydroxyanisole (BHA), butylated hydroxytoluene (BHT), lecithin, propyl gallate, and alpha-tocopherol; and (3) metal chelating agents, such as citric acid, ethylenediamine tetraacetic acid (EDTA), tartaric acid, and phosphoric acid;
antibacterial and antifungal agents, such as paraben, chlorobutanol, phenol, sorbic acid;
mucosal adhesion enhancers such as starch graft copolymers (e.g., starch/acrylic acid copolymers) and other water-swellable polymers that adhere to wet surfaces of the oral mucosa such as carbomers, hydrolysed polyvinyl alcohol, polyethylene oxides, and polyacrylates;
as well as other non-toxic compatible substances employed in pharmaceutical formulations, such as liposomes and micelle forming agents;
including mixtures thereof.

Preferred rapidly infusing compositions are those which contain less than 1 wt. %, preferably less than 0.5 wt. %, preferably less than 0.1 wt. %, preferably less than 0.05 wt. %, preferably less than 0.001 wt. %, preferably 0 wt. %, of other pharmaceutically acceptable carriers and/or excipients, such as those listed above, in particular alkaline buffering agents and/or surfactants.

Also preferred are rapidly infusing compositions which do not contain inert diluents, aqueous carriers, or non-aqueous carriers commonly used in the art for manufacture of liquid dosage forms for oral administration, such as emulsions, microemulsions, solutions, suspensions, syrups, and elixirs. Examples of inert diluents, aqueous or non-aqueous carriers, etc. which are preferably excluded herein may include, but are not limited to, water or other solvents, solubilizing agents, and emulsifiers, such as ethyl alcohol, isopropyl alcohol, ethyl carbonate, ethyl acetate, benzyl alcohol, benzyl benzoate, glycerol, polyethylene glycol, propylene glycol, 1,3-butylene glycol, oils (whether synthetic, semi-synthetic, or naturally occurring, such as long chain triglycerides, mixed glycerides, and free fatty acids, in particular, cottonseed oil, groundnut oil, corn oil, germ, olive oil, castor oil, sesame oil, borage oil, coconut oil, soybean oil, safflower oil, sunflower oil, palm oil, peanut oil, peppermint oil, poppy seed oil, canola oil, hydrogenated soybean oil, hydrogenated vegetable oils, glyceryl distearate, behenic acid, caprylic/capric glycerides, lauric acid, linoleic acid, linolenic acid, myristic acid, palmitic acid, palmitoleic acid, palmitostearic acid, ricinoleic acid, stearic acid, soy fatty acids, oleic acid, glyceryl esters of fatty acids such as glyceryl behenate, glyceryl isostearate, glyceryl laurate, glyceryl palmitate, glyceryl palmitostearate, glyceryl ricinoleate, glyceryl oleate, glyceryl stearate), tetrahydrofuryl alcohol, fatty acid esters of sorbitan, organic esters such as ethyl oleate, and mixtures thereof, with specific mention being made to ethyl alcohol and sesame oil.

### Active Therapeutic Ingredient (ATI)

The amount of active therapeutic ingredient (ATI) which can be combined with the pharmaceutically acceptable carrier and/or excipient system to produce the rapidly infusing composition may vary depending upon the subject being treated, and other factors. The amount of ATI which can be combined with the pharmaceutically acceptable carrier and/or excipient system to produce a single dosage form will generally be that amount which produces a therapeutic effect. Generally, this amount will range from 0.1 to 9) wt. % of ATI, for example, at least 20 wt. %, preferably at least 22 wt. %, preferably at least 24 wt. %, preferably at least 26 wt. %, preferably at least 28 wt. %, preferably at least 30 wt. %, preferably at least 32 wt. %, preferably at least 34 wt. %, preferably at least 36 wt. %, preferably at least 38 wt. %, preferably at least 40 wt. %, preferably at least 42 wt. %, preferably at least 44 wt. %, preferably at least 46 wt. %, preferably at least 48 wt. %, preferably at least 50 wt. %, preferably at least 52 wt. %, preferably at least 54 wt. %, and up to 70 wt. %, preferably up to 68 wt. %, preferably up to 66 wt. %, preferably up to 64 wt. %, preferably up to 62 wt. %, preferably up to 60 wt. %, preferably up to 58 wt. %, preferably up to 56 wt. % of the ATI, based on a total weight of the rapidly infusing composition on a dry basis.

In terms of unit dose, the rapidly infusing composition is generally formulated with 2 to 100 mg of ATI per unit (e.g. tablet), for example at least 2 mg, preferably at least 4 mg, preferably at least 6 mg, preferably at least 8 mg, preferably at least 10 mg, preferably at least 12 mg, preferably at least 14 mg, preferably at least 16 mg, preferably at least 18 mg, preferably at least 20 mg, preferably at least 22 mg, preferably at least 24 mg, and up to 100 mg, preferably up to 75 mg, preferably up to 70 mg, preferably up to 65 mg, preferably up to 60 mg, preferably up to 55 mg, preferably up to 50 mg, preferably up to 45 mg, preferably up to 40 mg, preferably up to 35 mg, preferably up to 30 mg, preferably up to 25 mg of ATI per unit (e.g., tablet).

In preferred embodiments, the rapidly infusing composition is formulated with, as the active therapeutic ingredient, cannabidiol (CBD). In some preferred embodiments, CBD is the only active therapeutic ingredient in the rapidly infusing composition. In other embodiments, CBD may be combined with other active therapeutic ingredients.

Preferred rapidly infusing compositions are those which are formulated with CBD, preferably a solid form of CBD. That is, the rapidly infusing composition is prepared through lyophilization from a drug product suspension in which the CBD is in the form of a solid. In particular, micronized particles of CBD are preferred. In some embodiments, the rapidly infusing composition is formulated with solid CBD in the form of micronized particles having a D50 particle size in the range of 1 μm to 50 μm, for example, those having a D50 particle size of at least 1 μm, preferably at least 10 μm, preferably at least 20 μm, preferably at least 30 μm, preferably at least 40 μm, and up to 50 μm, preferably up to 40 μm, preferably up to 30 μm, preferably up to 20 μm, preferably up to 10 μm.

Even more preferred are those rapidly infusing compositions which are formulated with a solid form of CBD having a purity of at least 95 wt. %, preferably at least 96 wt. %, preferably at least 97 wt. %, preferably at least 98 wt. %, preferably at least 99 wt %. While CBD having a purity of 100 wt. % is likely not achievable, preferably rapidly infusing compositions are formulated with a solid form of CBD having a purity up to 99.1 wt. %, preferably up to 99.2 wt. %, preferably up to 99.3 wt. %, preferably up to 99.4 wt. %, preferably up to 99.5 wt %, preferably up to 99.6 wt. %, preferably up to 99.7 wt. %, preferably up to 99.8 wt. %, preferably up to 99.9 wt. %. The percent purity of CBD refers to the percent of CBD by mass relative to a total weight of CBD containing material—the CBD containing material being the sum of CBD plus any additional impurities which may be present, such as those impurities originating from the biomass from which the CBD is obtained (e.g., Cannabis sativa L./“Industrial Hemp”) or encountered during manufacture. The purity may be determined by methods known to those of ordinary skill in the art, for example, one or more of liquid chromatography such as high performance liquid chromatography (HPLC), liquid chromatography-mass spectrometry (LCMS), and liquid chromatography with tandem mass spectrometry (LCMSMS); gas chromatography such as headspace gas chromatography with flame ionization detection (HS-GC-FID), gas chromatography mass spectrometry (GC/MS), and headspace gas chromatography-mass spectrometry (HSGCMS); inductively coupled plasma-mass spectrometry (ICP-MS); and polymerase chain reaction (PCR).

Examples of potential impurities, such as those originating from the biomass from which the CBD is obtained (e.g., Cannabis sativa L./“Industrial Hemp”) or encountered during manufacture, include, but are not limited to,
cannabinoids (other than CBD) including, but not limited to, cannabidivarin (CBDV), cannabichromene (CBC), cannabidiolic acid (CBDa), cannabigerol (CBG), cannabigerolic acid (CBGa), cannabinol (CBN), tetrahydrocannabinolic acid (THCa), tetrahydrocannabivarin (THCV), tetrahydrocannabivarin acid (THCVa), and tetrahydrocannabinol (Δ9-THC) and related THC-cannabinoids such as Δ8-THC;
pesticides including, but not limited to, aldicarb, carbofuran, chlordane, chlorfenapyr, chlorpyrifos, coumaphos, daminozide, dichlorvos (DDVP), dimethoate, ethoprophos, etofenprox, fenoxycarb, fipronil, imazalil, methiocarb, methyl parathion, paclobutrazol, propoxur, spiroxamine, and thiacloprid;
residual solvents including, but not limited to, 1,4-dioxane, 2-butanol, 2-ethoxyethanol, 1,2-dichloroethane, acetone, acetonitrile, benzene, butane, cumene, cyclohexane, chloroform, ethanol, ethyl acetate, ethyl benzene, ethylene oxide, ethylene glycol, ethyl ether, heptane, isopropanol, methanol, methylene chloride, hexanes, isopropyl acetate, pentanes, propane, toluene, tetrahydrofuran, trichloroethene, and xylenes;
microbials including, but not limited to, Aspergillus flavus, Aspergillus fumigatus, Aspergillus niger, Aspergillus terreus, Salmonella, and Shiga toxin-producing E. coli;
mycotoxins including, but not limited to, aflatoxins (e.g., aflatoxin B1, aflatoxin B2, aflatoxin G1, and aflatoxin G2) and ochratoxin A;
heavy metals including, but not limited to, arsenic, cadmium, lead, and mercury;
terpenes including, but not limited to, (1) monoterpenes such as camphene, camphor, 3-carene, α-cedrene, cedrol, endo-fenchyl alcohol, eucalyptol, fenchone, geraniol, geranul acetate, hexahydrothymol, isoborneol, isopulegol, limonene, linalool, p-mentha-1,5-diene, β-myrcene, α- and β-pinene, pulegone, sabinene and hydrate, α- and γ-terpinene, terpineol, terpinolene, α-, β-, and γ-terpineol, nerol, borneol, and ocimene isomers I and II, and (2) sesquiterpenes such as α-bisabolol, β-caryophyllene, caryophyllene oxide, guaiol, α-humulene, cis- and trans-nerolidol, and valencene;
as well as mixtures thereof.

In some embodiments, the rapidly infusing composition is formulated with a form of CBD which contains less than 1 wt. %, preferably less than 0.5 wt. %, preferably less than 0.1 wt. %, preferably less than 0.05 wt. %, preferably less than 0.001 wt. %, preferably 0 wt. % of the above listed impurities, based on a total weight of the CBD material, with specific mention being made to THC. In some embodiments, the rapidly infusing composition is formulated with a form of CBD which contains no impurity, such as those listed above, in an amount above the limits of detection (LOD) and/or limits of quantification (LOQ) for the technique/instrumentation being used to make such a determination. For example, preferred rapidly infusing compositions are those formulated with a pure form of CBD which has a THC content of less than 0.1577 wt. %, preferably less than 0.1 wt. %, preferably less than 0.01 wt. %, preferably less than 0.001 wt. %, based on a total weight of the CBD material. In preferred embodiments, the rapidly infusing composition is formulated with a pure form of CBD which consists of, or consists essentially of, CBD.

The full effects of the present disclosure may not be realized when the rapidly infusing composition is formulated with an impure form of CBD or when the composition is formulated with CBD in oil/liquid form. Without being bound by theory, it is believed that during the manufacture of the rapidly infusing composition, when the CBD is in solid form with sufficiently high purity, lyophilization from a drug product suspension generates a structured and robust matrix of gelatin as the water is removed via sublimation, and an even distribution of the CBD throughout the gelatin matrix. Such a structured assembly of CBD suspended within a gelatin matrix is believed to afford the rapidly infusing composition with rapid disintegration properties and efficient transfer of CBD from the hydrophilic vehicle to the mucous membrane of the buccal cavity, or the ventral surface under the tongue, upon administration.

On the contrary, when the composition is formulated with an impure (oil) form of CBD during manufacture, lyophilization is instead performed from an o/w emulsion of CBD, which may produce an unstable, disordered matrix of gelatin more prone to collapse back into an oil or semi-solid state. The resulting composition tends to suffer from poor shelf-life, increased disintegration times, and inferior delivery/uptake of the CBD into systemic circulation reflected in longer onset times and overall less efficacy against COVID-19.

Accordingly, any CBD manufacturing method known by those of ordinary skill in the art which provides CBD in solid form, and of sufficient purity, may be utilized herein for preparation of the CBD ATI. For illustration purposes, one exemplary CBD manufacturing method is described below, although it should be understood that numerous modifications and variations are possible, and the CBD may be produced using methods or techniques otherwise than as specifically described.

CBD may be extracted/isolated from biomass, for example, a cured flower of Cannabis sativa L. The biomass may contain, for example, at least 1 mg/g, preferably at least 2 mg/g, preferably at least 3 mg/g, and up to 10 mg/g, preferably up to 8 mg/g, preferably up to 6 mg/g, preferably up to 4 mg/g of CBD; at least 50 mg/g, preferably at least 60 mg/g, preferably at least 70 mg/g, preferably at least 80 mg/g, preferably at least 90 mg/g, and up to 150 mg/g, preferably up to 140 mg/g, preferably up to 130 mg/g, preferably up to 120 mg/g, preferably up to 110 mg/g, preferably up to 100 mg/g of cannabidiolic acid (CBDa); and no detectable amount of THC. Extraction of the biomass with an alcoholic solvent (e.g., ethanol) and cooling may form a tincture. The tincture may be filtered to remove sediment and particulates, and concentrated, for example, using a rotary evaporator.

An aluminum phyllosilicate clay (e.g., bentonite) may then be mixed with the concentrated product at a weight ratio of at least 2:1, preferably at least 3:1, preferably at least 4:1, and up to 6:1, preferably up to 5:1, and the resulting mix filtered to remove fats, waxes, and lipids. The product may then be frozen/winterized, after which the frozen product may be again filtered and taken through another solvent removal/recovery cycle to form a winterized crude.

Decarboxylation of the winterized crude by heating, for example in an induction oven centrifugal reactor, may be performed to remove the carboxylic acid functionality from the cannabinoids. Distillation of the decarboxylated material may then provide a distillate.

The distillate may then be precipitated in a high-pressure reactor using an alkane solvent (e.g., pentane), and a cryochamber may be used to subject the precipitate to cryo temperatures (e.g., −20° F. to −40° F.) to promote the growth of crystalline CBD. The CBD crystals may be washed with an alkane solvent (e.g., pentane), filtered, and ground to a finer particle size, prior to being purged in a vacuum oven for removal of solvents and impurities. The obtained solid CBD may then be analyzed for purity, as appropriate.

In preferred embodiments, the rapidly infusing composition comprises, consists essentially of, or consists of gelatin, mannitol, sweetener, flavorant, colorant, and as the ATI, CBD.

Also contemplated for use as an active therapeutic ingredient are derivatives/analogs of CBD that retain the desired activity for the treatment of other medical conditions. Derivatives/analogs that retain substantially the same activity as CBD, or more preferably exhibit improved activity, may be produced according to standard principles of medicinal chemistry, which are well known in the art. Such derivatives/analogs may exhibit a lesser degree of activity than CBD, so long as they retain sufficient activity to be therapeutically effective. Derivatives/analogs may exhibit improvements in other properties that are desirable in active therapeutic agents such as, for example, improved solubility, reduced toxicity, enhanced uptake, increased bioavailability, etc. Contemplated CBD derivatives/analogs include, but are not limited to, cannabidiolic acid compounds and variants thereof, such as cannabidiolic acid and esters of cannabidiolic acid, in particular alkyl esters of cannabidiolic acid (e.g., cannabidiolic acid methyl ester); 5′ side chain modified CBD compounds such as cannabidivarin (CBDV), cannabidiol-dimethylheptyl (CBD-DMH), and 1,2-cannabidiol-dimethylheptyl (1,2-CBD-DMH); 7-methyl modified CBD compounds such as 7-carboxy cannabidiol (7-COOH-CBD) and 7-hydroxy cannabidiol (7-OH-CBD); hydrogenated CBD compounds such as 8,9-dihydrocannabidiol (H₂-CBD) and tetrahydrocannabidiol (H₄-CBD); halogenated CBD compounds such as 3′-chloro-CBD, 3′,5′-dichloro-CBD, 3′-bromo-CBD, 3′,5′-dibromo-CBD, 3′-iodo-CBD, and 3′,5′-diiodo-CBD; hydroxyl group modified CBD compounds such as desoxy-CBD and dimethylether CBD; cannabielsoin (CBE); machaeridiols A, B, and C; as well as any pharmaceutically acceptable salts, solvates, and/or stereoisomers of such compounds. When a CBD derivative/analog is used as the ATI in the disclosed rapidly infusing composition, particular preference is given to 7-hydroxy cannabidiol (7-OH-CBD). It is contemplated that CBD or derivatives/analogs of CBD may be useful in combination. Specific dosages and dosing regimens would be based on physicians' evolving knowledge and the general skill in the art.

### Process for Manufacturing the Rapidly Infusing Composition

Manufacturing of the rapidly infusing compositions are preferably pharmaceutical-GMP compliant and may be accomplished generally by bringing into association the ATI (e.g., CBD) with the gelatin and sugar alcohol (e.g., mannitol), and, optionally, one or more accessory pharmaceutically acceptable carrier and/or excipient ingredients, in water to form a drug product suspension which is then lyophilized.

One exemplary method for manufacturing the rapidly infusing composition is presented below, although it should be understood that numerous modifications and variations are possible, and the rapidly infusing composition may be produced using methods or techniques otherwise than as specifically described.

Purified water, gelatin, and sugar alcohol (e.g., mannitol) may be charged to a mixer, for example a pot equipped with an overhead stirrer, and heated (e.g., 40 to 80° C.) with agitation until complete solvation. Any desired sweetener (e.g., a mixture of sucralose and acesulfame-K) may then be added and allowed to dissolve.

Upon cooling, for example to 20 to 35° C., the solution may next be transferred to a homogenizer, and the ATI (e.g., CBD) may be subsequently charged and dispersed using the homogenizer, with preferable micronization of the ATI, to form a drug product suspension. Any desired flavorant and colorant may be added at this point with continued mixing. The drug product suspension may be transferred to a second mixer whilst maintaining a cooled temperature (e.g., 20 to 35° C.).

In a blistering machine equipped with a dosing system, blister pockets may next be filled with the drug product suspension until achieving a target dose weight, followed by freezing in a suitable cryochamber. The blister trays may be transferred from the cryochamber to a suitable refrigerated storage cabinet (e.g., at a temperature below 0° C.) to keep the product frozen prior to lyophilization. Then, the frozen blisters may be loaded into a lyophilizer and subject to lyophilization to sublimate the water and form the rapidly infusing compositions. Finally, when the lyophilization cycle is deemed complete, final sealing (e.g., heat sealing of blister lidding) may be performed to provide the rapidly infusing compositions in single dose units in individual blister units.

### Therapeutic Applications and Methods

The present disclosure provides a method of treating COVID-19 by administering to a subject in need thereof the disclosed rapidly infusing composition, in one or more of its embodiments.

In one embodiment, the subject presents with a common symptom of COVID-19, including but not limited to ageusia (loss of taste), anosmia (loss of smell), cough, diarrhea, fatigue, fever, headache, migraine headache, joint pain, muscle pain, nasal congestion, parosmia (change in sense of smell), runny nose, shortness of breath, and sore throat.

In one embodiment, the subject may present with a positive test for SARS-CoV-2. The test may be an antigen test or a nucleic acid test, such as a reverse transcription polymerase chain reaction test (RT-PCR test). In a related embodiment, the subject is asymptomatic, presenting with a positive test for SARS-CoV-2 infection but not exhibiting a symptom associated with COVID-19. However, an asymptomatic subject may later develop COVID-19 symptoms.

In one embodiment, the rapidly infusing composition may have an efficacy that varies depending on the strain or variant of the SARS-CoV-2 causing the infection. The strain or variant may be Alpha (lineage B.1.1.7), B.1.1.7 with E484K, Beta (lineage B.1.351), Gamma (lineage P.1), Delta (lineage B.1.617.2), Omicron (lineage B.1.1.529), Lambda (lineage C.37), Mu (lineage B.1.621, Epsilon (lineages B.1.429, B.1.427, CAL.20C), Zeta (lineage P.2), Eta (lineage B.1.525), Theta (lineage P.3), Iota (lineage B.1.526), Kappa (lineage B.1.617.1). Preferably the SARS-CoV-2 strain or variant is considered a variant of concern by the World Health Organization (WHO), such as Alpha, Beta, Gamma, Delta, and Omicron. In an alternative embodiment, the rapidly infusing composition may be used to treat an infection by a coronavirus that is not SARS-CoV-2.

Administration of the rapidly infusing composition may result in a decrease in the severity or frequency of COVID-19 symptoms. A decrease in the severity or frequency of COVID-19 symptoms may be by at least 50%, preferably at least 60%, preferably at least 70%, preferably at least 75%, preferably at least 80%, preferably at least 85%, preferably at least 90%, preferably at least 95%, preferably at least 99%, preferably 100%, relative to the frequency or severity observed prior to administration of the rapidly infusing composition.

In one embodiment, the administration of the rapidly infusing composition may result in a decrease in the average length of a hospital stay, for instance by at least 10%, preferably at least 20%, preferably at least 25%, preferably at least 40%, preferably at least 50%, preferably at least 60%, preferably at least 75%.

In one embodiment, administration of the rapidly infusing composition may result in a decrease in the replication of the SARS-CoV-2 in a subject. For instance, the administration may result in a viral load or virus titer that is decreased by at least 2-fold, preferably at least 3-fold, preferably at least 4-fold, preferably at least 5-fold, preferably at least 8-fold, preferably at least 10-fold, relative to the viral load prior to administration. Here, the viral load may be measured from nasal secretions, for instance, collected from nasal turbinate by a nasopharyngeal swab. The viral load measured from lung secretions may show a greater decrease, for instance, at least 10-fold, preferably at least 20-fold, preferably at least 30-fold, preferably at least 40-fold, as compared to the viral load in lung secretions prior to administration.

In a related embodiment, the administration of the rapidly infusing composition to a subject who tests positive for SARS-CoV-2 may result in a decrease in the number of days they continue to test positive by nucleic acid and/or antigen test. For instance, the subject may first test negative for SARS-CoV-2 at least 1 day earlier, preferably at least 2 days earlier, preferably at least 3 days earlier, preferably at least 4 days earlier, preferably at least 5 days earlier than if they had not been administered the rapidly infusing composition.

In a related embodiment, the administration of the rapidly infusing composition to a subject who tests positive for SARS-CoV-2 and presents with a fever (defined here as having a body temperature of 100.4° F. (38° C.) or greater) may result in a decrease in the duration of the fever by at least 1 day, preferably at least 2 days, preferably at least 3 days, preferably at least 4 days, preferably at least 5 days. By ameliorating COVID-19 symptoms with the administration of the rapidly infusing composition, including decreasing the duration of a fever, a subject presenting COVID-19 may be able to decrease the length of their self-isolation period by at least 1 day, preferably at least 2 days, preferably at least 3 days, preferably at least 4 days, preferably at least 5 days, preferably at least 6 days, preferably at least 7 days.

In one embodiment, the subject is administered the rapidly infusing composition at an early stage of SARS-CoV-2 infection, for instance, 5 days or less from infection, the onset of COVID-19 symptoms, or from receiving a positive test result, preferably 4 days or less, preferably 3 days or less, preferably 2 days or less, preferably 1 day or less. However, the subject may be administered the rapidly infusing composition at a later time and still show improvement.

In one embodiment, administration of the rapidly infusing composition may be used to treat long COVID, also known as Post-Acute Sequelae of SARS-CoV-2 infection (PASC). Long COVID occurs when symptoms of COVID persist more than four weeks after initial infection, and some symptoms may persist for months. These symptoms include anosmia, blood clotting (deep vein thrombosis and pulmonary embolism), changes in mood, changes in oral health (teeth, gums, saliva), chest pains, diarrhea and bouts of vomiting, extreme fatigue, gastroesophageal reflux disease, headaches, inability to concentrate (brain fog), joint pain, kidney problems, long lasting cough, low grade fever, memory lapses, muscle weakness, needle pains in arms and legs, new onset of diabetes and hypertension, palpitations, parosmia, shortness of breath, skin rash, sleep difficulties, sore throat and difficulties swallowing, and tinnitus. In one embodiment, administration of the rapidly infusing composition may begin at the onset of COVID-19 symptoms or with indication of a positive SARS-CoV-2 test, and may decrease the severity of long COVID symptoms. Alternatively, administration may not begin until four weeks or more after the onset of COVID-19 symptoms or after a positive SARS-CoV-2 test, and yet may still decrease the severity of long COVID symptoms.

Therapeutic methods may also include prophylactic or preventative therapies. In one embodiment the rapidly infusing composition is administered to a host who has been exposed to and thus is at risk of infection or at risk of reinfection of SARS-CoV-2. Prophylactic treatment may be administered, for example, to a subject not yet exposed to or infected with SARS-CoV-2 but who is susceptible to, or otherwise at risk of exposure or infection with SARS-CoV-2. In one embodiment, a person at risk for infection or reinfection is administered the rapidly infusing composition indefinitely until the risk of exposure no longer exists. In another related embodiment, a person who is asymptomatic may be administered the rapidly infusing composition following a known exposure to SARS-CoV-2, for instance, following contact with a person who has COVID-19 or following contact with a person who tested positive or later tests positive for SARS-CoV-2. Here, the ATI may inhibit viral replication of SARS-CoV-2 to prevent a full-scale infection. This inhibition of viral replication may decrease the intensity of COVID-19 symptoms or prevent COVID-19 entirely.

Without being bound by theory, it is hypothesized that CBD and/or one of its metabolites, 7-hydroxy-cannabidiol (7-OH-CBD), inhibit viral replication in cells by targeting host cell processes. CBD and/or 7-OH-CBD may prevent or inhibit the viral RNA expression of SARS-CoV-2 within the host cells; this may be achieved by promoting degradation of the viral RNA by activating IRE1α and interferon pathways. CBD and/or 7-OH-CBD may be more effective at blocking viral replication at early stages of infection, or more effective as a prophylactic treatment, as compared to inhibiting an infection at a later stage.

In another alternative embodiment, a method to prevent transmission is provided that includes administering the rapidly infusing composition described herein for a sufficient length of time prior to and after exposure to crowds that can be infected, including during travel or public events or meetings, including for example, up to 1, 2, 3, 5, 7, 10, 12, 14 or more days prior to and after a communicable situation, either because the person is infected or to prevent infection from an infected person in the communicable situation.

With respect to administration, the rapidly infusing composition is preferably administered to the subject via one or more of the oral mucosae, preferably via the buccal mucosa (buccally) or the sublingual mucosa (sublingually). Advantages of oral mucosal delivery include the ease of administration, the ability to bypass first pass metabolic processes thereby enabling higher bioavailability than through enteral delivery via the gastrointestinal tract (which in turn allows the dosage amount of ATI to be reduced whilst maintaining the same pharmacological effect), less variability between patients, sustained drug delivery, and extensive drug absorption and rapid onset of therapeutic action due to either a large surface area in the case of sublingual administration or high-levels of vascularization in the case of buccal administration. Administration may be carried out by simply placing the rapidly infusing composition directly in the buccal cavity (between the cheek and gum) or over the sublingual mucous gland (under the ventral surface of the tongue). While the sublingual mucosa has a large surface area and extremely good permeability, the blood supply (blood flow) is lesser than that of the buccal cavity. Furthermore, sublingual administration tends to stimulate the flow of saliva more than buccal administration, and the increased saliva production may make it more difficult for patients to avoid swallowing. Any amount of ATI that is swallowed would be subject to first pass metabolism and thus overall lower bioavailability. Swallowing further results in greater variability in the effective amount of dosing, as a result of, including but not limited to, the variability in the amount swallowed and the greater patient variability of bioavailability through first-pass metabolism for the amount swallowed. Therefore, in preferred embodiments, the rapidly infusing composition is administered buccally (through the buccal mucosa). The rapid disintegration of the rapidly infusing composition, approximately in 1-5 seconds in preferred embodiments, and buccal administration together combine to provide optimal dosing control by limiting the time for potential swallowing and ensuring that the vast majority of the ATI is absorbed through the buccal mucosa. Administration may be performed by the subject (self-administered) or by someone other than the subject, for example, a healthcare provider, care-taker, family member, etc.

The actual amount of ATI administered to the subject may be varied so as to achieve the desired therapeutic response for a particular subject, composition, and mode of administration, without being toxic to the subject. The selected amount of ATI administered to the subject will depend upon a variety of factors including the condition being treated, the activity of the ATI employed, the route of administration, the time of administration, the rate of excretion or metabolism of the particular compound being employed, the rate and extent of absorption, the duration of the treatment, other drugs, compounds, and/or materials used in combination with the rapidly infusing composition, the age, sex, weight, condition, general health, and prior medical history of the subject being treated, and like factors well known in the medical arts.

A physician having ordinary skill in the art can readily determine and prescribe the effective amount of the ATI required. For example, the physician could start doses of the ATI at levels lower than that required in order to achieve the desired therapeutic effect and gradually increase the dosage until the desired effect is achieved. In general, a suitable dose of the ATI will be that amount which is the lowest dose effective to produce a therapeutic effect, which will generally depend upon the factors described above. Typically, when the ATI is CBD or a derivative/analog thereof, the therapeutically effective amount of CBD or a derivative/analog thereof will be under 7 mg/kg/day, for example in a range of from at least 0.1 mg/kg/day, preferably at least 0.15 mg/kg/day, preferably at least 0.2 mg/kg/day, preferably at least 0.25 mg/kg/day, preferably at least 0.3 mg/kg/day, preferably at least 0.35 mg/kg/day, preferably at least 0.4 mg/kg/day, preferably at least 0.45 mg/kg/day, preferably at least 0.5 mg/kg/day, preferably at least 0.55 mg/kg/day, preferably at least 0.6 mg/kg/day, and up to 6.9 mg/kg/day, preferably up to 6.8 mg/kg/day, preferably up to 6.6 mg/kg/day, preferably up to 6.4 mg/kg/day, preferably up to 6.2 mg/kg/day, preferably up to 6 mg/kg/day, preferably up to 5.5 mg/kg/day, preferably up to 5 mg/kg/day, preferably up to 4.5 mg/kg/day, preferably up to 4 mg/kg/day, preferably up to 3.5 mg/kg/day, preferably up to 3 mg/kg/day, preferably up to 2.5 mg/kg/day, preferably up to 2 mg/kg/day, preferably up to 1.5 mg/kg/day, preferably up to 1.25 mg/kg/day, preferably up to 1 mg/kg/day, preferably up to 0.9 mg/kg/day, preferably up to 0.8 mg/kg/day, preferably up to 0.7 mg/kg/day, preferably up to 0.65 mg/kg/day.

In one embodiment, the therapeutically effective amount of CBD will be 7 mg/kg/day or less, preferably up to 6.5 mg/kg/day, preferably up to 6 mg/kg/day, preferably up to 5.5 mg/kg/day, preferably up to 5 mg/kg/day.

In a preferred embodiment, CBD is the only active therapeutic ingredient in the rapidly infusing composition, and the rapidly infusing composition is substantially free of other CBD derivatives and CBD analogs. Here, that the composition has a content of CBD derivatives and analogs of less than preferably less than 0.1 wt. %, preferably less than 0.01 wt. %, preferably less than 0.001 wt. %, or about 0 wt. % based on a total weight of the CBD. In a further embodiment, the CBD is the only active therapeutic ingredient in the rapidly infusing composition and the subject is not administered a cannabinoid other than CBD (for instance, by co-administration).

The methods herein may involve administering one, or more than one, unit of the rapidly infusing composition per dose (dosing event). For example, in circumstances where each unit of the rapidly infusing composition contains 25 mg of ATI (e.g., CBD), and it has been determined that a subject weighing 75 kg requires a therapeutically effective amount of 2 mg/kg/day of ATI, then the subject may be given two (2) units (e.g., tablets) three times a day (t.i.d.) to achieve the desired therapeutically effective amount of 2 mg/kg/day. In another example, in circumstances where each unit of the rapidly infusing composition contains 12.5 mg of ATI (e.g., CBD), and it has been determined that a subject weighing 75 kg requires a therapeutically effective amount of 2 mg/kg/day of ATI, then the subject may be given four (4) units (e.g., tablets) three times a day (t.i.d.) to achieve the desired therapeutically effective amount of 2 mg/kg/day. Accordingly, depending on the unit dose of ATI in each unit of the rapidly infusing composition, the therapeutically effective amount of ATI prescribed, etc., 1, 2, 3, 4, 5, or more units (e.g., tablets) may be administered to the subject per dose. Accordingly, the phrases “administering to the subject in need thereof a rapidly infusing composition”, “the rapidly infusing composition is administered”, etc., are intended herein to include administration of a single unit (e.g., tablet), or multiple units (e.g., tablets), to the subject in order to provide the therapeutically effective amount of ATI, e.g., CBD. While it may be possible to administer partial (e.g., half) tablets to the subject, for practical reasons, it is preferred that one or more whole tablets are administered to the subject.

In preferred embodiments, the subject may be prescribed a dosage regimen that involves a single dosing event per day (QD), or multiple, separate dosing events at appropriate time intervals throughout the day. The subject may be administered a therapeutically effective amount of ATI 1 time, 2 times, 3 times, 4 times, 5 times, 6 times, 7 times, 8 times, 9 times, or 10 times at appropriate intervals, throughout the day. Preferred dosing regimens involve administration at the same time each day, for example, following meal times every morning and/or evening. Particularly preferred dosing schedules involve administration of the rapidly infusing composition once (QD), two times (b.i.d.), or three times (t.i.d.) per day. The rapidly infusing composition may also be administered on an hourly dosing schedule (q), for example, administration may take place every 8 to 12 hours, as appropriate. Regardless of dosing schedule, when the ATI is CBD, the maximum daily dosage of CBD is preferably less than 7 mg/kg/day. Treatment may involve administration until desired effects are achieved, for example for weeks, months, or even years, or throughout the subjects life-span.

Preferred dosing regimens are those involving a consistent dosing amount and schedule. One non-limiting example of a dosing regimen may involve the subject taking one unit of the rapidly infusing composition (e.g., 25 mg CBD)—therapeutically effective amount of 25 mg CBD per dose—once per day (QD). Another non-limiting example of a dosing regimen may involve the subject taking one unit of the rapidly infusing composition (e.g., 25 mg CBD)—therapeutically effective amount of 25 mg CBD per dose—two times per day (b.i.d.). Another non-limiting example of a dosing regimen may involve the subject taking two units of the rapidly infusing composition (e.g., 10 mg CBD each)—therapeutically effective amount of 20 mg CBD per dose—two times per day (b.i.d.).

Upon being administered buccally (between the cheek and gum) or sublingually (under the ventral surface of the tongue), the rapidly infusing composition preferably disintegrates in 5 seconds or less, preferably 4 seconds or less, preferably 3 seconds or less, preferably 2 seconds or less, preferably about 1 second. Further, this route of administration may provide a single dose bioavailability of at least 20%, preferably at least 30%, preferably at least 40%, preferably at least 50%, preferably at least 60%, preferably at least 70%, preferably at least 80%, preferably at least 90%, and up to 95%, preferably up to 90%, preferably up to 80%, preferably up to 70%, preferably up to 60%, preferably up to 50%, preferably up to 40%, preferably up to 30%.

Besides efficacy of treatment and general relief from COVID-19 symptoms, pharmacokinetic outcomes may provide another useful measure of in vivo performance. In this regard, the rapidly infusing composition formulated with CBD and administered according to the methods described herein may provide a time to maximum plasma concentration (Tmax) of less than 5 hours, preferably less than 4 hours, preferably less than 3 hours, preferably less than 2 hours, preferably less than 1 hour, preferably less than 45 minutes, preferably less than 30 minutes, preferably less than 15 minutes; an area under the plasma concentration versus time curve (AUC) of at least 1 h×ng/mL, preferably at least 3 h×ng/mL, preferably at least 5 h×ng/mL, preferably at least 10 h×ng/mL, preferably at least 15 h×ng/mL, preferably at least 20 h×ng/mL, preferably at least 25 h×ng/mL, preferably at least 30 h×ng/mL, and up to 80 h×ng/mL, preferably up to 70 h×ng/mL, preferably up to 60 h×ng/mL, preferably up to 50 h×ng/mL, preferably up to 40 h×ng/mL, from a single (1) unit of rapidly infusing composition formulated with 25 mg CBD; and a mean plasma half-life (t_{1/2}) of CBD of at least 1 hour, preferably at least 2 hours, preferably at least 3 hours, preferably at least 4 hours, preferably at least 5 hours, preferably at least 6 hours, and up to 12 hours, preferably up to 11 hours, preferably up to 10 hours, preferably up to 9 hours, preferably up to 8 hours, preferably up to 7 hours, for a single dose, but may provide a significantly higher mean plasma half-life (t_{1/2}) after prolonged buccal or sublingual administration (e.g., t_{1/2} of 2 to 5 days).

Using the platform, the rapidly infusing composition may be used as a stand-alone therapeutic agent for the treatment of COVID-19 or may be used in combination therapy—wherein the rapidly infusing composition is used in combination with one or more other forms of therapy such as one or more second therapeutic agents. The combination therapy may be applied to treat COVID-19, or a combination of COVID-19 and a different condition.

Combination therapy may involve administering the rapidly infusing composition formulated with CBD and a second therapeutic agent, other than CBD for the treatment of COVID-19. In particular, when co-administered with one or more antiviral drugs, the rapidly infusing compositions formulated with CBD may advantageously function to decrease antiviral usage, for example by reducing the number of antivirals dosed, reducing the overall dosage of the antivirals, or shortening the dosage period, without sacrificing the drug's effect.

An antiviral drug may be administered with the rapidly infusing composition. Examples of antiviral drugs include, but are not limited to, amiodarone, artesunate, chlorpromazine, clemastine, elacridar, favipiravir, lopinavir, lopinavir/ritonavir, molnupiravir, nirmatrelvir, pyronaridine, remdesivir, ribavirin, ritonavir, sertraline, triazavirin, and umifenovir.

The antiviral drug may be an RNA virus antiviral drug, a DNA virus antiviral drug, and/or an antiretroviral drug. In a preferred embodiment, the antiviral drug is an RNA virus antiviral drug, including but not limited to adapromine, amantadine, asunaprevir, baloxavir marboxil, beclabuvir, bemnifosbuvir, boceprevir, bulevirtide, ciluprevir, CMX521, daclatasvir, daclatasvir, danoprevir, dasabuvir, deleobuvir, eicar, elbasvir, elbasvir, faldaprevir, favipiravir, filibuvir, galidesivir, glecaprevir, glecaprevir, grazoprevir, grazoprevir, GS-441524, GS-6620, IDX-184, interferon alfa 2b, laninamivir, ledipasvir, ledipasvir, lufotrelvir, mericitabine, merimepodib, MK-608, molnupiravir, moroxydine, narlaprevir, nirmatrelvir. NITD008, odalasvir, ombitasvir, ombitasvir, oseltamivir, paritaprevir, paritaprevir, peginterferon alfa-2a, peginterferon alfa-2b, peramivir, pibrentasvir, pibrentasvir, pimodivir, pleconaril, presatovir, radalbuvir, ravidasvir, remdesivir, ribavirin, rimantadine, ritonavir, ruzasvir, samatasvir, setrobuvir, simeprevir, sofosbuvir, sofosbuvir, sofosbuvir, sofosbuvir, sofosbuvir, sovaprevir, taribavirin, tegobuvir, telaprevir, TMC-647055, triazavirin, umifenovir, uprifosbuvir, valopicitabine, vaniprevir, vedroprevir, velpatasvir, velpatasvir, velpatasvir, voxilaprevir, voxilaprevir, and zanamivir. In one embodiment, the antiviral drug is an antiretroviral drug including, but not limited to, the protease inhibitors amprenavir, atazanavir, darunavir, fosamprenavir, indinavir, lopinavir, nelfinavir, ritonavir, saquinavir, tipranavir, and TMC-310911. In one embodiment, the antiviral drug is favipiravir, lopinavir/ritonavir, and/or remdesivir.

In another embodiment, the rapidly infusing composition may be administered with a monoclonal antibody. The monoclonal antibody may be bamlanivimab, bamlanivimab/etesevimab, baricitinib, casirivimab, casirivimab/imdevimab, cilgavimab, etesevimab, imdevimab, lenzilumab, regdanvimab, sarilumab, sotrovimab, tixagevimab, tixagevimabrcilgavimab, tixagevimab/cilgavimab, and/or tocilizumab.

In another embodiment, the rapidly infusing composition may be administered with an angiotensin-converting-enzyme (ACE) inhibitor. The ACE inhibitor may be alacepril, benazepril, captopril, ceronapril, cilazapril, delapril, enalapril, fosinopril, imidapril, lisinopril, moexipril, perindopril, quinapril, ramipril, rentiapril, spirapril, temocapril, trandolapril, and/or zofenopril.

In another embodiment, the rapidly infusing composition may be administered with an angiotensin-converting-enzyme (ACE) inhibitor. The ACE inhibitor may be alacepril, benazepril, captopril, ceronapril, cilazapril, delapril, enalapril, fosinopril, imidapril, lisinopril, moexipril, perindopril, quinapril, ramipril, rentiapril, spirapril, temocapril, trandolapril, and/or zofenopril.

In another embodiment, the rapidly infusing composition may be administered with an angiotensin II receptor blocker (ARB). The ARB may be azilsartan, candesartan, eprosartan, fimasartan, irbesartan, losartan, olmesartan, tasosartan, telmisartan, and/or valsartan.

In another embodiment, the rapidly infusing composition may be administered with some other drug or compound, such as an antibiotic including, but not limited to, teicoplanin, oritavancin, dalbavancin, monensin, azithromycin; a protease inhibitor such as CLpro-1, GC376, and rupintrivir; convalescent plasma; tamoxifen; sildenafil; vitamin D, including cholecalciferol and calcifediol; a histamine H2 receptor antagonist such as cimetidine or famotidine; a steroid including but not limited to dexamethasone, hydroconisone, budesonide, and ciclesonide; an antiparasitic such as chloroquine, hydroxychloroquine, mefloquine, ivermectin, and atovaquone; vitamin C; creatine; and an analgesic including but not limited to ibuprophen, acetaminophen, acetylsalicylic acid (aspirin), and naproxen.

Combination therapy is intended to embrace administration of these therapies in a sequential manner, that is, wherein the rapidly infusing composition and one or more other therapies are administered at a different time, as well as administration of these therapies, or at least two of the therapies, in a substantially simultaneous manner. Substantially simultaneous administration can be accomplished, for example, by administering to the subject multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, the rapidly infusing composition formulated with CBD may be administered via buccal administration while a second therapeutic agent of the combination may be administered intravenously.

Combination therapy also can embrace the administration of the rapidly infusing composition in further combination with other biologically active ingredients and non-drug therapies. Examples of non-drug therapies may include, but are not limited to, oxygen support, noninvasive ventilation, and mechanical ventilation. Where the combination therapy further comprises a non-drug treatment, the non-drug treatment may be conducted at any suitable time so long as a beneficial effect from the co-action of the combination of the therapeutic agent(s) and non-drug treatment is achieved. For example, in appropriate cases, the beneficial effect is still achieved when the non-drug treatment is temporally removed from the administration of the therapeutic agents, perhaps by days or even weeks.

The examples below are intended to further illustrate the materials and methods of the present disclosure, and are not intended to limit the scope of the claims.

Where a numerical limit or range is stated herein, the endpoints are included. Also, all values and subranges within a numerical limit or range are specifically included as if explicitly written out.

As used herein the words “a” and “an” and the like carry the meaning of “one or more.”

The present disclosure also contemplates other embodiments “comprising”, “consisting of” and “consisting essentially of”, the embodiments or elements presented herein, whether explicitly set forth or not.

All patents and other references mentioned above are incorporated in full herein by this reference, the same as if set forth at length.

Obviously, numerous modifications and variations of the present invention are possible in light of the above teachings. It is therefore to be understood that, within the scope of the appended claims, the invention may be practiced otherwise than as specifically described herein.

### EXAMPLES

### Rapidly Infusing Composition

### Ingredients

The ingredients that were used to make the rapidly infusing composition and the placebo are given in Table 1. USP=United States Pharmacopeia. EP=European Pharmacopoeia. NF=National Formulary.

TABLE 1
Ingredients

| Ingredient | Primary Function | Specification |
|---|---|---|
| Gelatin | Matrix former | USP/EP/NF |
| Mannitol | Bulking agent | USP/EP |
| Lemon-lime flavor powder | Flavorant | Non-compendial |
| CBD isolate | ATI | Non-compendial |
| Sucralose | Sweetener | USP/NF |
| Acesulfame-K | Sweetener | USP/NF |
| FD&C Yellow #5 | Colorant | Non-compendial |
| Purified water | Vehicle | USP/EP |

An example rapidly infusing composition was made using the formulation given in Table 2. The amount of each component is expressed in terms of weight percentage relative to a total weight (100%). The weight percentage of each component in the drug product suspension is on a wet basis (prior to removal of water). The weight percentage of each component in the rapidly infusing composition is on a dry basis (after removal of water).

TABLE 2
Example rapidly infusing composition formulation

| | Drug product | | | |
|---|---|---|---|---|
| | suspension | Rapidly Infusing Composition | | |
| | % wt./wt. | wt./unit | | % wt./wt. |
| Ingredient | (wet) | (dry) | | (dry) |
| Gelatin | 3.5 | 10.5 | mg | 22.7 |
| Mannitol | 3.0 | 9 | mg | 19.4 |
| Lemon-lime flavor | 0.2 | 0.6 | mg | 1.3 |
| powder | | | | |
| CBD isolate | 8.4 | 25 | mg | 54.0 |
| Sucralose | 0.2 | 0.6 | mg | 1.3 |
| Acesulfame-K | 0.2 | 0.6 | mg | 1.3 |

| | | | |
|---|---|---|---|
| FD&C Yellow #5 | Trace | Trace | Trace |
| Purified water | 84.5 | Removed during | Removed during |
| | | manufacture | manufacture |
| Total | 100.0 | — | 100.0 |

### Methods of Making the Rapidly Infusing Composition

Purified water was charged to a pot and mixed using an overhead stirrer as an agitating device.
With agitation, the requisite amount of gelatin and mannitol were dispersed, and the mixture was heated until the excipients were dissolved.
Once dissolved, the sweeteners sucralose and acesulfame-K were added and allowed to dissolve.
The solution was cooled to 30° C., moved to an overhead homogenizer, and then the requisite amount of cannabidiol (CBD) isolate was charged and dispersed using the homogenizer to micronize the CBD and create a drug product suspension.
The requisite amount of Lemon-Lime flavor was charged and mixed for 10 minutes, then the FD&C Yellow #5 colorant was added.
The resulting drug product suspension was transferred to a second overhead mixer and maintained at a temperature of 30° C. for the ensuing dosing operation.
In a blistering machine equipped with a dosing system, blister pockets were filled with a target dose weight of 300.0 mg of the drug product suspension.
The product was frozen in a suitable cryochamber and then the blister trays were transferred from the cryochamber to a suitable refrigerated storage cabinet (temperature below 0° C.) prior to lyophilizing to keep the product frozen.
The frozen blisters were loaded from the refrigerated storage cabinet into lyophilizers and the product was lyophilized (water was sublimated) to form the rapidly infusing compositions.
When the lyophilizing cycle was completed, the rapidly infusing compositions were transferred from the lyophilizers to the blistering machine where the blister trays were heat sealed with lidding material. The resulting tablets are flat-topped circular units approximately 15 mm in diameter with a convex bottom packaged in individual blister units (see also U.S. Provisional Application 63/114,181-incorporated herein by reference in its entirety).

The following tests were performed:
A seal integrity test was performed at −0.5 Bar for 30 seconds, 1-minute soak time
Visual inspection was performed
Dry weight testing was performed

### Placebo

A placebo product was also formulated in the same manner as the rapidly infusing composition, with the exception that the placebo product was formulated without CBD.

### Clinical Trial Results

Final results from a clinical trial addressing post-surgical pain following shoulder arthroscopy, the methodology for which is described in U.S. patent application Ser. No. 17/225,738 filed Apr. 8, 2021, which is incorporated herein by reference, show that the rapidly infusing dosage form of the instant invention was shown to be safe and effective in reducing pain and improving patient satisfaction in the immediate peri-operative period following arthroscopic rotator cup repair (ARCR).

On the safety issue, there were no serious adverse events reported among the participants in the experimental cohort, and only five (5) mild adverse events in total that could not confidently be attributed to CBD. This extremely low rate of adverse events, 9.4%, is basically unheard of in CBD trials and will likely make a tremendous difference in adherence to the therapy by future patients.

By contrast, clinical trial data for Epidiolex®, the only currently FDA-approved drug containing CBD, showed a far higher incidence of adverse events. For example, in epilepsy-related trials, adverse event rates varied from 45% (NCT02224703 at “mid dose” of 20 mg/kg/day) to 94% (NCT02564952 at 20-30 mg/kg/day) and a Parkinson's disease related trial (NCT02818777) experienced adverse events in 100% of the subjects. Results for pain-related clinical trials of Sativex®, an oromucosal spray containing both THC and CBD (which is not approved for any indication in the United States), also showed far more adverse events, with serious adverse events occurring in as high as 45.6% of patients (NCT01337089) and other adverse events (not including serious adverse events) occurring in as high as 97% of patients (NCT01606176) in certain trials.

1. A method for the treatment of COVID-19 caused by SARS-CoV-2 infection in a subject, the method comprising:
administering to the subject in need thereof, via the oral mucosa, a rapidly infusing composition comprising (a) a pharmaceutically acceptable binder and/or excipient system comprising gelatin and a sugar alcohol, and (b) a therapeutically effective amount of cannabidiol (CBD).

2. The method of claim 1, wherein the rapidly infusing composition is lyophilized.

3. The method of claim 1, wherein the rapidly infusing composition has a disintegration time of approximately 1 to 30 seconds in deionized water maintained at 37° C. 2° C.

4. The method of claim 1, wherein the rapidly infusing composition has a disintegration time of approximately 1 to 5 seconds in deionized water maintained at 37° C. 2° C.

5. The method of claim 1, wherein the gelatin is present in the rapidly infusing composition in an amount of 10 to 35 wt. %, based on a total weight of the rapidly infusing composition on a dry basis.

6. The method of claim 1, wherein the gelatin is bovine gelatin.

7. The method of claim 1, wherein the sugar alcohol is present in the rapidly infusing composition in an amount of 5 to 35 wt. %, based on a total weight of the rapidly infusing composition on a dry basis.

8. The method of claim 1, wherein the sugar alcohol comprises mannitol.

9. The method of claim 1, wherein the CBD is present in the rapidly infusing composition in an amount of 20 to 70 wt. %, based on a total weight of the rapidly infusing composition on a dry basis.

10. The method of claim 1, wherein the rapidly infusing composition is formulated with a solid form of the CBD.

11. The method of claim 1, wherein the rapidly infusing composition is formulated with a solid form of the CBD having a purity between 95 and 99.9 wt. %.

12. The method of claim 1, wherein the rapidly infusing composition is formulated with a solid form of the CBD that has been micronized to have a D50 diameter between 1 and 50 μm.

13. The method of claim 1, wherein the rapidly infusing composition further comprises at least one selected from the group consisting of a sweetener, a flavorant, and a colorant.

14. The method of claim 13, wherein the rapidly infusing composition comprises the flavorant, and the flavorant comprises lemon-lime flavor.

15. The method of claim 13, wherein the rapidly infusing composition comprises the colorant, and the colorant comprises FD&C Yellow ##5.

16. The method of claim 13, wherein the rapidly infusing composition comprises the sweetener, and the sweetener comprises a mixture of sucralose and acesulfame-K.

17. The method of claim 1, wherein the rapidly infusing composition is administered to the subject via the buccal mucosa.

18. The method of claim 1, wherein the therapeutically effective amount of CBD is from 0.1 mg/kg/day to less than 7 mg/kg/day.

19. The method of claim 1, wherein the therapeutically effective amount of CBD is from 0.1 mg/kg/day to less than 5 mg/kg/day.

20. The method of claim 1, wherein the rapidly infusing composition is administered to the subject 1 to 3 times per day.

21. The method of claim 1, wherein CBD is the only active therapeutic ingredient in the rapidly infusing composition.

22. The method of claim 21, wherein the subject is not administered a cannabinoid other than CBD.

23. The method of claim 1, wherein the subject presents with at least one symptom selected from the group consisting of ageusia, anosmia, cough, diarrhea, fatigue, fever, headache, migraine headache, joint pain, muscle pain, nasal congestion, parosmia, runny nose, shortness of breath, and sore throat.

24. The method of claim 1, wherein the subject presents with a positive nucleic acid test for SARS-CoV-2.

25. The method of claim 1, wherein the subject presents with a positive antigen test for SARS-CoV-2.

26. The method of claim 1, wherein the subject is asymptomatic.

27. The method of claim 1, wherein the subject is first administered the rapidly infusing composition at a time that is 5 days or less from the start of SARS-CoV-2 infection or 5 days or less from receiving a positive test result indicating SARS-CoV-2 infection.

28. The method of claim 1, wherein the subject is first administered the rapidly infusing composition at a time that is 3 days or less from the onset of a COVID-19 symptom.

29. The method of claim 1, wherein the subject has long COVID.

30. The method of claim 1, wherein the SARS-CoV-2 is a variant selected from the group consisting of Alpha, Beta, Gamma, Delta, and Omicron.

31. The method of claim 1, wherein a severity or a frequency of a COVID-19 symptom is reduced by at least 50%, relative to the frequency or severity observed prior to administration of the rapidly infusing composition.

32. The method of claim 1, wherein a viral load of the subject as measured by nasal secretion is reduced by at least 3-fold, relative to the viral load prior to administration of the rapidly infusing composition.

33. The method of claim 1, wherein a viral load of the subject as measured by lung secretion is reduced by at least 20-fold, relative to the viral load prior to administration of the rapidly infusing composition.

34. The method of claim 1, wherein the rapidly infusing composition is administered in combination with an antiviral drug.

35. The method of claim 34, wherein the antiviral drug is at least one selected from the group consisting of amiodarone, artesunate, chlorpromazine, clemastine, elacridar, favipiravir, lopinavir, molnupiravir, nirmatrelvir, pyronaridine, remdesivir, ribavirin, ritonavir, sertraline, triazavirin, and umifenovir.

36. The method of claim 34, wherein the antiviral drug is an RNA virus antiviral drug.

37. The method of claim 36, wherein the RNA virus antiviral drug is at least one selected from the group consisting of adapromine, amantadine, asunaprevir, baloxavir marboxil, beclabuvir, bemnifosbuvir, boceprevir, bulevirtide, ciluprevir, CMXS21, daclatasvir, daclatasvir, danoprevir, dasabuvir, deleobuvir, eicar, elbasvir, elbasvir, faldaprevir, favipiravir, filibuvir, galidesivir, glecaprevir, glecaprevir, grazoprevir, grazoprevir, GS-441524, GS-6620, IDX-184, interferon alfa 2b, laninamivir, ledipasvir, ledipasvir, lufotrelvir, mericitabine, merimepodib, MK-608, molnupiravir, moroxydine, narlaprevir, nirmatrelvir, NfTDO08, odalasvir, ombitasvir, ombitasvir, oseltamivir, paritaprevir, paritaprevir, peginterferon alfa-2a, peginterferon alfa-2b, peramivir, pibrentasvir, pibrentasvir, pimodivir, pleconaril, presatovir, radalbuvir, ravidasvir, remdesivir, ribavirin, rimantadine, ritonavir, ruzasvir, samatasvir, setrobuvir, simeprevir, sofosbuvir, sofosbuvir, sofosbuvir, sofosbuvir, sofosbuvir, sovaprevir, taribavirin, tegobuvir, telaprevir, TMC-647055, triazavirin, umifenovir, uprifosbuvir, valopicitabine, vaniprevir, vedroprevir, velpatasvir, velpatasvir, velpatasvir, voxilaprevir, voxilaprevir, and zanamivir.

38. The method of claim 1, wherein the rapidly infusing composition is administered in combination with an angiotensin-converting-enzyme inhibitor.

39. The method of claim 38, wherein the angiotensin-converting-enzyme inhibitor is at least one selected from the group consisting of alacepril, benazepril, captopril, ceronapril, cilazapril, delapril, enalapril, fosinopril, imidapril, lisinopril, moexipril, perindopril, quinapril, ramipril, rentiapril, spirapril, temocapril, trandolapril, and zofenopril.

40. The method of claim 1, wherein the rapidly infusing composition is administered in combination with a monoclonal antibody.

41. The method of claim 40, wherein the monoclonal antibody is at least one selected from the group consisting of bamlanivimab, bamlanivimab/etesevimab, baricitinib, casirivimab, casirivimab/imdevimab, cilgavimab, etesevimab, imdevimab, lenzilumab, regdanvimab, sarilumab, sotrovimab, tixagevimab, tixagevimab/cilgavimab, tixagevimabicilgavimab, and tocilizumab

42. The method of claim 1, wherein the rapidly infusing composition is administered in combination with an angiotensin II receptor blocker.

43. The method of claim 42, wherein the angiotensin II receptor blocker is at least one selected from the group consisting of azilsartan, candesartan, eprosartan, fimasartan, irbesartan, losartan, olmesartan, tasosartan, telmisartan, and valsartan.

44. The method of claim 1, wherein the subject is asymptomatic and had been exposed to SARS-CoV-2 before the administering.
